(19) United States
(12) Patent Application Publication
(10) Pub. No.: US 2023/0330294 A1
(43) Pub. Date: Oct. 19, 2023

(54) Odorant capsule and adaptable olfactory composition carrier for a device for emitting such a composition, kit for emitting an olfactory composition and its use

(71) Applicant: Enzo Didi, san francisco, CA (US)

(72) Inventor: Enzo Didi, san francisco, CA (US)

(21) Appl. No.: 18/340,190

(22) Filed: Jun. 23, 2023

(51) Int. Cl. A61L 9/012; A61L 9/12; A61L 9/04

(52) U.S. Cl. CPC A61L 9/012; A61L 9/12; A61L 9/042; A61L 2209/134

(57) ABSTRACT

The invention concerns at least one odorant capsule (20A), (20B), (20C) comprising at least one carrier compound (23) of an olfactory composition adaptable and usable in a device for emitting such an olfactory composition configured to vary the air flow generated by each of the air flow generating systems and a kit for emitting an olfactory composition.

### TECHNICAL AREA

The invention relates to the field of providing olfactory ambience in living spaces. The invention relates more particularly to at least one odorant capsule comprising at least one carrier compound of an olfactory composition adaptable and usable in a device for emitting such an olfactory composition configured to vary the air flow generated by each of the air flow generation systems.

### STATE OF THE ART

It is known that living spaces can be enhanced by the use of devices for emitting an olfactory composition based on sprays comprising a cartridge of a mixture of perfume and propellant gas. While it is possible to vary the olfactory ambience of one's interior by changing the cartridge or by using 2 or 3 devices in combination or alternately, the number of ambiences accessible remains limited and requires active intervention by the user to change ambience.

Thus, there would be merit in an olfactory composition emission device that allows easy adaptation of the latter without active intervention, such as a cartridge change, by the user.

### EXPLANATION OF THE INVENTION

The invention aims to remedy the above drawback and thus has the object of providing at least one odorant capsule comprising at least one carrier compound of an olfactory composition adaptable and usable in an emission device configured to vary the air flow generated by each of the air flow generation systems; the latter being able to allow the emission of an olfactory composition which can be adapted without active intervention by the user, i.e. without having to change a cartridge or trigger another emission device.

It will be noted that in the context of the present document, if the terminology of “emission” of the olfactory composition is used, such terminology is used in order to cover an active diffusion of the various compounds included in said olfactory composition. It is noted that in the present invention, the physical phenomenon enabling such emission is a forced diffusion of the various compounds provided by the passage of air flows along the air flow path of corresponding capsules.

A first object of the invention relates to a perfume capsule comprising at least one carrier compound of the olfactory composition made from a material limiting interactions between the capsule wall and the carrier compound of the olfactory composition. In one embodiment, the capsule is made from a plastic material that is resistant to chemicals selected from acids, alcohols, hydrocarbons, oils, particularly hot oils, and bases. In a preferred embodiment, the plastic material of the capsule is resistant to chemicals selected from Acetamide 50%, concentrated or diluted acetic acid, acetone, a 10% ammonia solution, anone, benzene, benzine, bitumen, aqueous boric acid solution, butyl acetate, 10% calcium chloride solution, carbon tetrachloride, chlorobenzene, chloroform, 10% aqueous citric acid solution, 50% clophene A60, cupric sulfate, cyclohexane, cyclohexanone, decalin, diesel, dimethyl formamide, dioctyl phthalate, methyl ethyl ketone, nitrobenzene, kerosene oil, ozone, aqueous oxalic acid, perchloroethylene, petroleum, aqueous solution of phenol, aqueous solution of phosphoric acid, concentrated phosporic acid, aqueous solution of potassium dichromate, salicylic acid, silicone oils, propanol, 1% aqueous solution of potassium permanganate.

It should be noted that in the context of the present invention, each carrier compound of the olfactory composition which is contained in a capsule serves as a carrier for a single olfactory compound (which may correspond to a basic flagrance) of the olfactory composition. The carrier compound is referred to as the olfactory composition carrier compound, since it serves as a carrier for each of the olfactory compounds of said olfactory composition.

According to a preferred embodiment of the invention, the capsule is made of polytetrafluoroethylene (PTFE). The capsule has a substantially cylindrical shape.

Another object of the invention relates to a capsule comprising at least one olfactory composition carrier compound. This olfactory composition carrier is made from a material chosen from plastics, clays, synthetic materials such as polyacrylamides and resins.

In one embodiment, the olfactory composition carrier compound is in the form of granules, spheres, pearls, beads or microbeads or equivalent shapes. Said olfactory composition carrier compound is intended for use in at least one capsule as described above.

A further object of the invention relates to a capsule insertable or intended to be inserted into a delivery device for an adaptable olfactory composition, said delivery device comprising: —at least three housings each designed to receive a respective capsule containing a possible compound of said olfactory composition in the form of solid granules, each capsule having an inlet face and an outlet face each apertured to define an air flow path between the inlet face and the outlet face, —for each housing, a variable airflow generation system to generate a variable airflow at said housing along the airflow path; the emission device being further configured to vary the airflow generated by each of the airflow generation systems in order to vary the proportion of olfactory compounds in the olfactory composition emitted by the emission device.

Another object of the invention concerns a combination product of a capsule and an olfactory composition carrier compound. In this combination product, interactions between the capsule wall and the olfactory composition carrier compound are reduced.

Another object of the invention relates to a kit for emitting an olfactory composition comprising: —a capsule as previously described, and at least one olfactory composition carrier compound as previously described.

A final object of the invention relates to the use of a capsule as previously described and/or a kit as described above and/or a combination product as previously defined, in an emission device for an adaptable olfactory composition, said emission device comprising: —at least three housings each intended to receive a respective capsule containing a possible olfactory compound of said olfactory composition in the form of solid granules, each capsule presenting an inlet face and an outlet face each apertured to define an air flow path between the inlet face and the outlet face, —for each housing, a variable airflow generation system to generate a variable airflow at said housing along the airflow path; the emission device being further configured to vary the airflow generated by each of the airflow generation systems in order to vary the proportion of olfactory compounds in the olfactory composition emitted by the emission device.

### BRIEF DESCRIPTION OF THE DRAWINGS

The present invention will be better understood on reading the description of example embodiments, given purely by way of illustration and in no way limiting, with reference to the appended drawings on which:

FIG. 1 illustrates the body of a device for emitting an olfactory composition after removal of the cover, the cartridges comprising the capsules showing the outlet faces each apertured to define an air flow path between the inlet face (not shown) and the outlet face.

FIG. 2 illustrates an olfactory composition delivery system comprising an olfactory composition delivery device and a mobile terminal for controlling said device.

FIG. 3 shows the capsule according to the invention, with the lid removed to reveal the olfactory composition's carrier compounds in the form of solid granules.

FIG. 4 illustrates the supporting compounds of the olfactory composition.

Identical, similar or equivalent parts of the various figures bear the same numerical references, so as to facilitate passage from one figure to another.

The various parts shown on the figures are not necessarily shown on a uniform scale, to make the figures easier to read.

The various possibilities (variants and realization modes) should be understood as not being mutually exclusive and can be combined with each other.

### A DETAILED DESCRIPTION OF SPECIFIC PRODUCTION METHODS

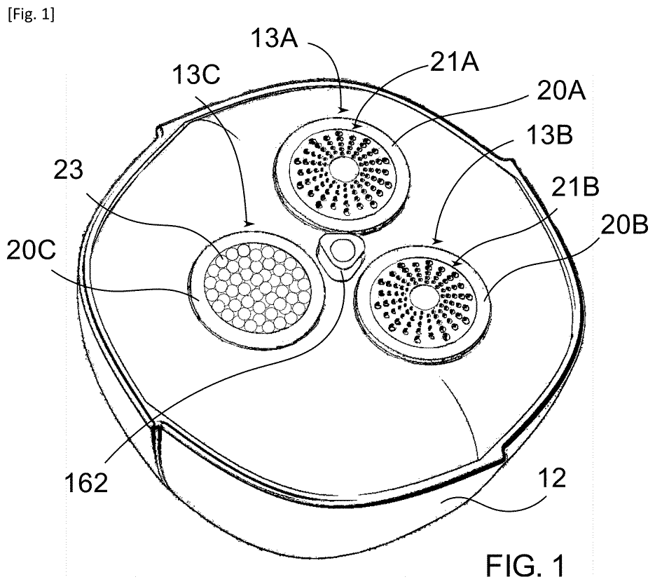

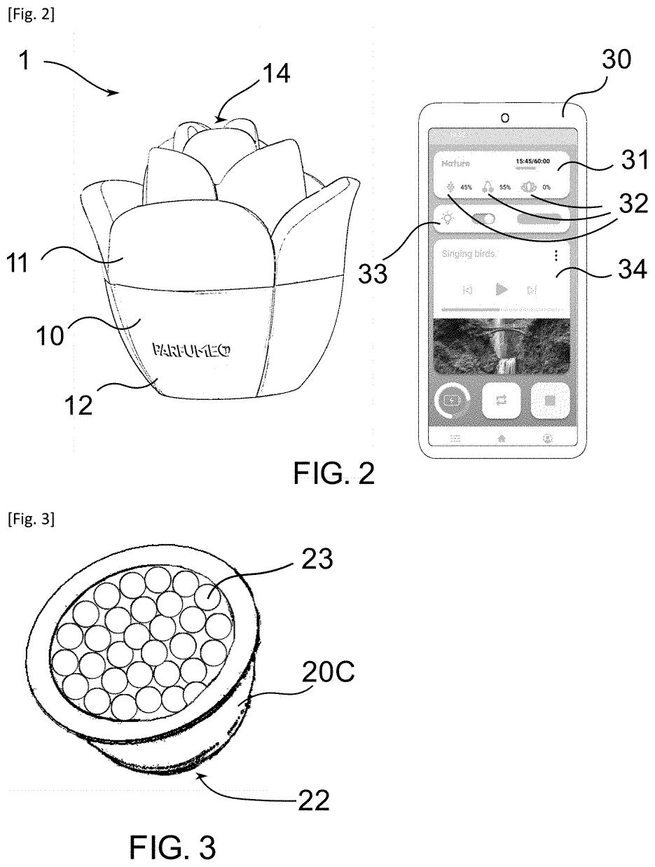

FIG. 1 shows the body (12) of a device (10) for dispensing an olfactory composition after removal of the cover (11), the device and cover being shown in FIG. 2, together with housings (13A), (13B) and (13C) into which capsules (20A, 20B, 20C) showing the outlet faces (22), visible on FIG. 3, each apertured in order to define an air flow path between the inlet face (21A) and (21B) and the outlet face (22), shown on FIG. 3.

In the context of the present invention, one embodiment involves an olfactory composition delivery system (1) as illustrated in FIG. 2 comprising the emission device (10). More specifically, the emission device (10) comprises: —a body (12) of the delivery device (10) having three housings (13A), (13B) and (13C) each receiving a respective capsule (20A), (20B), (20C) containing a possible carrier compound (23) of said olfactory composition, preferably in the form of solid granules, a lid of the third capsule (20C) having been removed to show said carrier compound (23), each capsule (20A), (20B), (20C) presenting an inlet face (21A), (21B), and an outlet face (22), —a lid (11) presenting an outlet face (22), and a lid (11) with an outlet (14) through which the olfactory composition is emitted.

It should be noted that the device (10) for emitting an adaptable olfactory composition is designed to be coupled to a mobile terminal (30) of the cell phone type or, more precisely, of the smart phone type (better known as a smartphone), in order to ensure its implementation and to form the delivery system (1) for the olfactory composition. As shown in FIG. 1, the capsules (20A), (20B) and (20C) are shaped to suit the housing. According to one embodiment, the shape of the capsules (20A), (20B) and (20C) is substantially cylindrical with a circular or oval base. Of course, such a shape is only an example of a particularly advantageous embodiment of the invention and will be adapted to the general shape of the transmitter device (10), which can take on a variety of shapes, such as cylindrical, parallelepipedal or, for decorative purposes, any human, animal or plant shape, without departing from the scope of the invention.

A first object of the invention is a perfume capsule (20C) illustrated in FIG. 3 comprising at least one carrier compound (23) for the olfactory composition. The capsule (20A), (20B) and (20C) is made of a material that limits interactions between the capsule wall and the olfactory composition carrier compound. In one embodiment, the capsules (20A), (20B) and (20C) are made of a plastic material. Generally speaking, this is a plastic material that is resistant to chemicals selected from acids, alcohols, hydrocarbons, oils (particularly hot oils) and bases.

In particular, the plastic material is resistant to chemicals selected from acetamide 50%, concentrated or dilute acetic acid, acetone, 10% ammonia solution, anone, benzene, benzine, bitumen, aqueous boric acid solution, butyl acetate, 10% calcium chloride solution, carbon tetrachloride, chlorobenzene, chloroform, 10% aqueous citric acid solution, 50% clophene A60, cupric sulfate, cyclohexane, cyclohexanone, decalin, diesel, dimethyl formamide, dioctyl phthalate, methyl ethyl ketone, nitrobenzene, kerosene oil, ozone, aqueous oxalic acid solution, perchloroethylene, petroleum, aqueous solution of phenol, aqueous solution of phosphoric acid, concentrated phosporic acid, aqueous solution of potassium dichromate, salicylic acid, silicone oils, propanol, 1% aqueous solution of potassium permanganate.

Preferably, the capsules are made from polytetrafluoroethylene (PTFE).

The invention concerns at least one odorant capsule (20A), (20B), (20C) comprising at least one carrier compound (23) of an olfactory composition and usable in a device for emitting such an olfactory composition capable of modulating the composition without changing the capsule (20A), (20B), (20C). In fact, said emission device (10) being further configured to vary the air flow generated by each of the air flow generating systems, in the present embodiment a fan not shown, in order to vary the proportion of olfactory compounds in the olfactory composition emitted by the emission device (10). With such a device, it is easy to adapt the olfactory composition by simply modulating the air flows generated by the air flow generation systems. It is thus easy to switch from an olfactory composition composed of 100% of an olfactory compound (32) of said composition of a given capsule, i.e. a single airflow generating system generating one airflow, to an olfactory composition composed of several olfactory compounds (32) varying in different proportions, preferably with two other capsules, this by stopping or reducing the single airflow generating system and switching on the other two airflow generating systems. Of course, in accordance with the invention, such variation does not require any active intervention on the part of the user, since it can, according to one possibility of the invention, be obtained by a simple software command from, for example, a mobile application as illustrated in FIG. 2. As mentioned in the examples, the distribution of the olfactory compounds (32) of said olfactory composition can vary from 0% to 100% each by combining the capsules (20A, 20B, 20C) with each other. In fact, via a dedicated application, as seen in FIG. 2, the user can define a program (31) for which a predefined olfactory composition is to be emitted, in this case an olfactory composition comprising the following olfactory compounds (32): 45% of a compound (32) corresponding to grapes, 55% of a compound (32) corresponding to cherries, and 0% of an olfactory compound (32) corresponding to lotus. The application also features a control button (33) for the lighting system of the broadcasting device (10), and a control button (34) for music broadcasting.

With regard to the capsule, the inlet face (21A) and (21B) and the outlet face (22) of the capsule (20A), (20B) and (20C) are each apertured, as shown in FIG. 1. The inlet face (21A) and (21B) of the capsule (20A) and (20B) thus have a plurality of circular apertures distributed along the radii of the central face around an aperture-free central circle. The openings have a diameter that increases with distance from the central circle. The output face (22) has two complementary metal contacts at its center (not shown in the figures). Around these two complementary metal contacts, the outlet face (22) has eight arched openings distributed over two circles, not shown, the outermost openings being larger than the innermost ones.

With such an opening in the inlet face (21A) and (21B) and the outlet face (22), the capsule defines an air flow path between the inlet face (21A) and (21B) and the outlet face (22). This airflow path allows air to circulate between the granules of the carrier compound, serving as a carrier for a respective olfactory compound, which is contained in the capsule (20A), (20B) and (20C).

FIG. 3 illustrates a capsule (20C) according to the invention, from which, in a similar way to the third capsule (20C) in FIG. 1, the lid has been removed to show the carrier compounds of the olfactory composition in the form of solid granules (23).

It is understood within the framework of the invention that the carrier compounds of the olfactory composition are in the form of granules, spheres, pearls, beads or microbeads or equivalent forms in a preferred mode of the invention; all being considered equivalent and interchangeable. It is also understood that all other shapes compatible with the dimensions of the capsules are also envisaged within the framework of the invention and with smaller or smaller sizes.

Preferably, the carrier compounds of the olfactory composition according to the invention have a diameter of between 0.2 cm and 1.5 cm; preferably between 0.4 cm and 1 cm. Even more preferably, the diameter is between 0.5 cm and 0.9 cm.

These olfactory composition carrier compounds (23) are in the form of granules or pearls or beads or spheres, the names being interchangeable, and can be placed in the capsules (20A and/or 20B and/or 20C) as defined above so as to fill them. The carrier compounds (23) of the olfactory composition can be colored.

The carrier compounds (23) of the olfactory composition in the above-mentioned forms are made of any materials suitable for impregnation with a fragrance or perfume. They can be made of natural materials such as clay, or synthetic materials such as polyacrylamide or resins. Preferably, this material is a resin.

In one embodiment, the olfactory composition carrier compounds in the above-mentioned forms are made of thermosetting synthetic resins such as phenolic resins. They have good dimensional stability, hardness and resistance to heat and corrosion. In particular, they are resistant to contact with weak acids and bases, hydrocarbons, petroleum products, alcohols, glycols, mineral oils and greases. Epoxy resins are also envisaged within the scope of the invention.

Preferably, the carrier compounds of the olfactory composition in the above-mentioned forms according to the invention are made of silicone, the main ingredient of which is Polysiloxalane/Polydimethylsiloxane. Silicone, sometimes known as polysyloxane, is a tetravalent inorganic element composed of a silicon-oxygen chain. When cross-linked with rubber, it becomes a high-performance elastomer.

Silicone beads have the advantage of being able to be used over a wide temperature range, without ever losing their properties. Optimum operating temperatures range from −65° C. to +180° C. They are highly resistant to contact with water (including hot water), oxygen, ozone, hydraulic fluids, animal and vegetable oils and fats, and diluted acids. Directly derived from silicon obtained from rock and sand, silicone is biocompatible. It is harmless to human cells and does not give off toxic fumes. Manufactured from a natural resource, its composition involves no animal matter. That's why this polymer is approved by the relevant authorities for the manufacture of food-grade silicone beads. As these silicone beads are widely used, the person skilled in the art will know how to obtain those of the invention.

The carrier compounds (23) of the olfactory composition in the above-mentioned forms according to the invention are impregnated and/soaked with olfactory compound or slowly diffused in such a way that partial degradation over time releases the olfactory compound from the olfactory composition under the effect of the passage of air generated by the fans of the emission device as described above.

The carrier compounds (23) of the olfactory composition according to the invention can be perfumed in the mass by the olfactory compound of the olfactory composition to obtain a constant fragrance from start to finish.

According to one embodiment, the olfactory compounds are made from pure natural extracts and/or essential oils. According to another embodiment, the olfactory compounds are synthesized. In particular, the olfactory compounds may comprise dimethylphenethyl butyrate. This ingredient is used as flavors and/or fragrances. From an organoleptic point of view, it provides a floral, herbaceous, fruity and/or green note. In the context of the invention, this ingredient is used alone or in combination, depending on the desired olfactory blend.

Another object of the invention is olfactory composition using blends. The scents diffused by means of the device described above enable fragrances to be mixed. Fragrances can be distributed using one, two or even three types of fragrance capsule. By way of example, the user of the device can choose, via a dedicated application, to define a program (31) for which a predefined olfactory composition is to be emitted. The distribution of the olfactory compounds (32) varies from 0 to 100% for each of them. For example, the olfactory composition comprising the following olfactory compounds (32) will comprise 45% of an olfactory compound (32) corresponding to grapes, 55% of an olfactory compound (32) corresponding to cherries, and 0% of an olfactory compound (32) corresponding to lotus. The application also features a control button (33) for the lighting system of the transmitting device (10), and a control button (34) for playing music. The scents making up the olfactory composition are very varied. Preferably, they will be chosen to bring harmony and well-being to the user. For example, scents chosen from: almond, aniseed, avocado, basil, chamomile, cherry, lemongrass, poppy, fig, passion fruit, pomegranate, jasmine, fresh herbs, kiwi, lavender, lilac, lotus, mango, lemon balm, mint, mimosa, coconut, passionflower, peach, periwinkle, pine, grape, tomato. Other scents or fragrances can also be used in the device according to the invention.

Advantageously, the technology within the scope of the invention releases the olfactory composition without the use of gas, additives or heat. In fact, with such a configuration of capsules, the air flow generated by the fan in the housing (13A), (13B), (13C) (not visible in the figures) will enable olfactory molecules of said carrier compound to be diffused and transported towards the opening in order to form the olfactory composition in the tube (162) (visible in FIG. 1) and enable it to be diffused at the opening (14).

According to the invention, at least one carrier compound (23) and preferably several carrier compounds (23) of the olfactory composition and at least one capsule (20A), or (20B), or (20C), preferably three capsules (20A), (20B), (20C), can form a kit for emitting olfactory compositions.

Another object of the invention concerns a combination product of a capsule (20A), (20B), (20C) and a carrier compound (23) of the olfactory composition. In this combination product, interactions between the wall of the capsule (20A), (20B), (20C) and the carrier compound (23) of the olfactory composition are reduced.

A final object of the invention relates to the use of a capsule (20A), (20B), (20C) as previously described and/or a kit as described above and/or a combination product as previously defined, in an emission device for an adaptable olfactory composition, said emission device comprising: —at least three housings each designed to receive a respective capsule (20A), (20B), (20C) containing a possible olfactory compound of said olfactory composition in the form of solid granules of the carrier compound (23), each capsule (20A, 20B, 20C) having an inlet face (21A) and (21B) and an outlet face (22) each perforated to define an air flow path between the inlet face (21A), (21B) and the outlet face (22), —for each housing (13A), (13B) and (13C), a variable airflow generation system to generate a variable airflow at said housing (13A), (13B) and (13C) along the airflow path; the emission device (10) being further configured to vary the airflow generated by each of the airflow generation systems in order to vary the proportion of olfactory compounds in the olfactory composition emitted by the emission device (10).

The invention is described in the examples by way of illustration and does not constitute a limitation of the invention.

### EXAMPLES

Example 1: Production of resin beads with a polymerizable monomer containing acrylic silicone copolymer: —a solution is prepared by dissolving a polyethylene glycol dimethacrylate with lauryl methacrylate and a non-polymerizable acrylic silicone copolymer, and the resulting mixture is mixed with a polymerizable monomer. a solution is prepared by dissolving polyethylene glycol dimethacrylate with lauryl methacrylate and a non-polymerizable acrylic silicone copolymer, and the resulting mixture is uniformly stirred and mixed to prepare a polymerizable monomer liquid. The acrylic silicone copolymer used above is preferably a graft polymer containing an acrylic polymer and a dimethylpolysiloxane; —in a further container, the polymerizable monomer liquid is mixed with the acrylic silicone copolymer. in another container, the polymerizable monomer liquid prepared above was added and mixed with an aqueous phase obtained by dissolving Polyvinyl Alcohol (PVA) as dispersion stabilizer in water, and the resulting mixture was stirred to obtain a uniform liquid suspension; —the resulting liquid suspension was then cast into molds which were placed in a polymerization apparatus. —After cooling, the resin beads/pearls produced were filtered and washed.

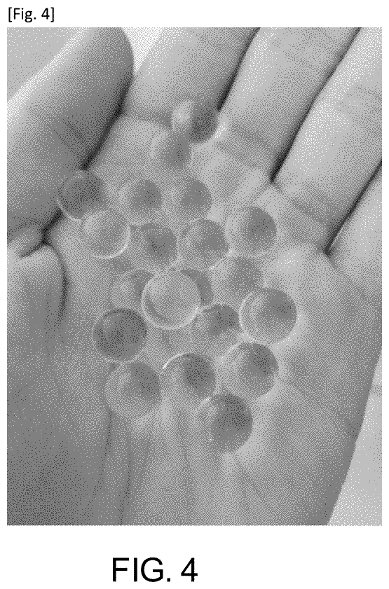

The resin beads are shown in FIG. 4 (photo).

Example 2: Examples of the distribution of fragrances or perfumes from olfactory compounds (32) impregnating olfactory composition carrier compounds 2a) example with 2 olfactory compounds (32): —45% to 55% of an olfactory compound (32) corresponding to grapes, —45% to 55% of an olfactory compound (32) corresponding to cherries, and—0% of an olfactory compound (32) corresponding to lotus.

2b) example with 3 olfactory compounds (32): —20% to 30% of an olfactory compound (32) corresponding to lotus—30% to 50% of an olfactory compound (32) corresponding to peach—20% to 50% of an olfactory compound (32) corresponding to grapes

2c) example of 3 olfactory compounds (32): —30% to 50% of an olfactory compound (32) corresponding to peach—20% to 50% of an olfactory compound (32) corresponding to grape—0 to 50% of an olfactory compound (32) corresponding to lavender

1. Perfume capsule (20A), (20B), (20C) comprising at least one olfactory composition carrier compound (23), characterized in that it is made of a material limiting interactions between the capsule wall and the olfactory composition carrier compound.

2. Capsule (20A), (20B), (20C) according to claim 1, characterized in that it is made of a plastic material resistant to chemicals chosen from acids, alcohols, hydrocarbons, oils and in particular hot oils and bases.

3. Capsule (20A), (20B), (20C) according to claim 2, characterized in that it is made of a chemical-resistant plastic material selected from acetamide 50%, concentrated or dilute acetic acid, acetone, a 10% ammonia solution, anone, benzene, benzine, bitumen, an aqueous boric acid solution, butyl acetate, a 10% calcium chloride solution, tetrachloroformaldehyde, tetrachloroformaldehyde, tetrachloroformaldehyde, tetrachloroformaldehyde, anone, benzene, benzine, bitumen, aqueous boric acid solution, butyl acetate, 10% calcium chloride solution, carbon tetrachloride, chlorobenzene, chloroform, 10% aqueous citric acid solution, 50% clophene A60, cupric sulfate, cyclohexane, cyclohexanone, decalin, diesel, dimethyl formamide, dioctyl phthalate, methyl ethyl ketone, nitrobenzene, kerosene oil, ozone, aqueous oxalic acid solution, perchloroethylene, petroleum, aqueous solution of phenol, aqueous solution of phosphoric acid, concentrated phosporic acid, aqueous solution of potassium dichromate, salicylic acid, silicone oils, propanol, 1% aqueous solution of potassium permanganate.

4. Capsule (20A), (20B), (20C) according to claim 3, characterized in that it is made of polytetrafluoroethylene (PTFE).

5. Capsule (20A), (20B), (20C) according to claim 1, characterized in that it comprises at least one olfactory composition support compound made from a material chosen from plastics, clays, synthetic materials such as polyacrylamides and resins.

6. Capsule (20A), (20B), (20C) according to claim 1, characterized in that it has a substantially cylindrical shape.

7. Capsule (20A), (20B), (20C) according to claim 1, characterized in that the carrier compound of the olfactory composition is in the form of granules, spheres, pearls, beads or microbeads or equivalent shapes.

8. Capsule (20A), (20B), (20C) according to any one of claims 1 to 6 insertable or intended to be inserted in a device for emitting (10) an adaptable olfactory composition, said device for emitting (10) comprising:
at least three housings (13A), (13B), (13C) each designed to receive a respective capsule (20A), (20B), (20C) containing a possible olfactory compound of said olfactory composition in the form of solid granules (23), each capsule (20A), (20B), (20C) having an inlet face (21A, 21B) and an outlet face (22) each perforated to define an air flow path between the inlet face (21A), (21B) and the outlet face (22),
for each housing (13A), (13B), (13C), a variable airflow generation system to generate a variable airflow at said housing (13A), (13B), (13C) along the airflow path; the emission device (10) being further configured to vary the airflow generated by each of the airflow generation systems in order to vary the proportion of olfactory compounds in the olfactory composition emitted by the emission device (10).

9. Carrier compound (23) for the olfactory composition, characterized in that it is made of a material selected from plastics, clays, synthetic materials such as polyacrylamides and resins.

10. Carrier compound (23) of the olfactory composition according to claim 9, characterized in that it is in the form of granules, spheres, pearls, beads or microbeads or equivalent shapes.

11. Kit for emitting an olfactory composition comprising:
A capsule (20A), (20B), (20C) according to claim 8
At least one carrier compound (23) of the olfactory composition according to claim 9.

12. Kit according to claim 11, characterized in that the carrier compound of the olfactory composition comprises at least one resin bead.

13. Use of a capsule according to claims 1 to 8 or of a kit according to claim 11, in an emission device (10) for an adaptable olfactory composition, said emission device (10) comprising:
at least three housings (13A), (13B), (13C), each designed to receive a respective capsule (20A), (20B), (20C) containing a possible olfactory compound of said olfactory composition in the form of solid granules (23), each capsule (20A), (20B), (20C) having an inlet face (21A), (21B) and an outlet face (22) each perforated to define an air flow path between the inlet face (21A), (21B) and the outlet face (22),
for each housing (13A), (13B), (13C), a variable airflow generation system to generate a variable airflow at said housing (13A), (13B), (13C) along the airflow path; the emission device (10) being further configured to vary the airflow generated by each of the airflow generation systems in order to vary the proportion of olfactory compounds in the olfactory composition emitted by the emission device (10).

14. Use of a carrier compound (23) of the olfactory composition according to claim 9 in at least one capsule (20A), (20B), (20C) according to claims 1 to 8.
